(19) United States
(12) Patent Application Publication
(10) Pub. No.: US 2025/0375309 A1
(43) Pub. Date: Dec. 11, 2025

(54) FLUID COLLECTION ASSEMBLIES DEFINING WAIST AND LEG OPENINGS

(71) Applicant: PUREWICK CORPORATION, Covington, GA (US)

(72) Inventor: Jill W. Jones, Atlanta, GA (US)

(21) Appl. No.: 19/240,380

(22) Filed: Jun. 17, 2025

Related U.S. Application Data
(63) Continuation of application No. 17/444,792, filed on Aug. 10, 2021, now Pat. No. 12,350,187.
(60) Provisional application No. 63/064,126, filed on Aug. 11, 2020.

(51) Int. Cl. A61F 5/44; A61F 5/451; A61M 1/00

(52) U.S. Cl. CPC A61F 5/4404; A61F 5/451; A61M 1/73; A61M 1/74

(57) ABSTRACT

Fluid collection assemblies and systems and methods of using the same are disclosed herein. An example fluid collection assembly includes a fluid impermeable barrier. The fluid impermeable barrier at least partially defines a waist opening and two leg openings. The waist opening and the two leg openings are configured to have a waist and two legs of an individual positioned therein, respectively. The fluid impermeable barrier also defines at least one fluid outlet. The fluid collection assembly further includes at least one porous material conforming to at least a portion of the fluid impermeable barrier. The porous material may be adjacent to the outlet defined by the fluid impermeable barrier.

### CROSS-REFERENCE FOR RELATED APPLICATION(S)

This application is a continuation of U.S. application Ser. No. 17/444,792 filed on Aug. 10, 2021, which claims priority to U.S. Provisional Patent Application No. 63/064,126 filed on Aug. 11, 2020, the disclosure of each of which is incorporated herein, in their entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of fluid collection devices continue to seek new and improved devices, systems, and methods to collect urine.

### SUMMARY

In an embodiment, a fluid collection assembly is disclosed. The fluid collection assembly including a fluid impermeable barrier at least partially defining a waist opening, two leg openings, and at least one fluid outlet. The fluid collection assembly also including at least one porous material generally conforming to at least a portion the fluid impermeable barrier.

In an embodiment, a fluid collection system is disclosed. The fluid collection assembly including a fluid impermeable barrier at least partially defining a waist opening, two leg openings, and at least one fluid outlet. The fluid collection assembly also including at least one porous material generally conforming to at least a portion the fluid impermeable barrier. The fluid collection system also includes a vacuum source configured to apply a suction force to the fluid collection assembly to withdraw one or more bodily fluids therefrom, a fluid storage container, and at least one conduit coupled to and extends between the at least one fluid outlet, the vacuum source, and the fluid storage container.

In an embodiment, a method of using a fluid collection assembly is disclosed. The method including receiving bodily fluids from an individual wearing the fluid collection assembly into at least one porous material of the fluid collection assembly. The fluid collection assembly includes a fluid impermeable barrier at least partially defining a waist opening, two leg openings, and at least one fluid outlet. The fluid collection assembly also includes at least one porous material that generally conforms to at least a portion the fluid impermeable barrier. The method also includes removing at least some of the bodily fluids from the fluid collection assembly.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is an isometric view of a fluid collection assembly, according to an embodiment.

FIG. 1B is a cross-sectional schematic of the fluid collection assembly taken along plane B-B shown in FIG. 1A, according to an embodiment.

FIG. 1C is a block diagram of a fluid collection system for fluid collection, according to an embodiment.

FIGS. 2A and 2B are cross-sectional views of fluid collection assemblies that include fluid reservoirs, according to different embodiments.

FIG. 3A is a cross-sectional schematic of a fluid collection assembly that includes a plurality of fluid outlets, according to an embodiment.

FIG. 3B is a block diagram of a fluid collection system that includes the fluid collection assembly illustrated in FIG. 3A, according to an embodiment.

FIG. 3C is a block diagram of a fluid collection system that includes the fluid collection assembly illustrated in FIG. 3A, according to an embodiment.

FIG. 4 is a cross-sectional schematic of a portion of a fluid collection assembly, according to an embodiment.

FIG. 5 is an isometric view of a fluid collection system that includes a portable vacuum source on an individual, according to an embodiment.

FIG. 6 is a flow diagram of a method to collect fluid, according to an embodiment.

### DETAILED DESCRIPTION

Embodiments disclosed herein are directed to fluid collection assemblies and systems and methods of using the same. An example fluid collection assembly includes a fluid impermeable barrier. The fluid impermeable barrier at least partially defines a waist opening and two leg openings. The waist opening and the two leg openings are configured to have a waist and two legs of an individual positioned therein, respectively. The fluid impermeable barrier also defines at least one fluid outlet. The fluid collection assembly further includes at least one porous material conforming to at least a portion of the fluid impermeable barrier. The porous material may be adjacent to the outlet defined by the fluid impermeable barrier.

The fluid collection assembly is configured to be worn by an individual. When worn by the individual, the fluid collection assembly is positioned on an individual such that the waist and legs of the individual are disposed in the waist opening and the leg openings, respectively. As such, the fluid collection assembly may be worn similar to some underwear (e.g., panties, boxer briefs, etc.). The porous material is positioned on the fluid collection assembly such that the porous material is adjacent to a urethral opening (e.g., vaginal region or penis) when the fluid collection assembly is worn by the individual. Positioning the waist and legs through the waist opening and the leg openings, respectively, represent an improvement over some conventional fluid collection assemblies. For example, some conventional fluid collection assemblies may require the individual to remain substantially still otherwise the convention fluid collection assemblies may become displaced from the urethral opening or required adhesives to applied to the individual which may cause pain when removed. However, disposing the waist and legs of the individual through the waist and leg openings maintains the position of the porous material of the fluid collection assemblies disclosed adjacent to the urethral opening even when the individual moves, such as when the individual changes position (e.g., switches between a standing, sitting, or sitting position), walks, runs, or otherwise moves. Further, the fluid collection assemblies disclosed herein may be able to store more bodily fluids therein compared to such conventional fluid collection assemblies (e.g., conventional fluid collection assemblies that do not define a waist opening and two leg openings).

The porous material may move the bodily fluids away from the urethral opening and towards the fluid outlet such that the bodily fluids may be removed from the fluid collection assembly. Generally, the porous material is formed from a wicking material and does not include an absorbent or adsorbent material since such wicking materials do not store the bodily fluids therein. As such, the wicking material draws the bodily fluids towards the fluid outlet more quickly than if the porous material included an adsorbent or absorbent material thereby allowing the porous material to receive a greater quantity of bodily fluids and at a greater rate of discharge than the adsorbent or absorbent material. Further, the wicking material may decrease the quantity of bodily fluids that are present in the fluid collection assembly a short period of time after the bodily fluids are discharged from the urethral opening thereby maintaining the skin of the individual drier than if the porous material included an adsorbent or absorbent material. The drier skin reduces the likelihood that the fluid collection assembly causes skin degradation (e.g., rashes) and encourages wound healing. However, it is noted that, in some embodiments, the porous material may include at least one adsorbent and/or absorbent material.

As previously discussed, the fluid collection assembly may form part of a system. The additional components of the system allow the system to remove bodily fluids from the fluid collection assembly through the fluid outlet. In other words, unlike conventional fluid collection assemblies such as diapers and pads, the fluid collection assembly is configured to allow and encourages the bodily fluids to flow out of the fluid collection assembly. Removing the bodily fluids from the fluid collection assembly through the fluid outlet allows the fluid collection assembly to receive more bodily fluids that other conventional fluid collection assemblies (e.g., diapers and pads) without needing to be replaced. Further, removing the bodily fluids from the fluid collection assembly allows the skin of the individual to remain drier compared to using the other conventional fluid collection assemblies. In an embodiment, the system may include a vacuum source and a fluid storage container. The vacuum source and the fluid storage container may be coupled to the fluid outlet via at least one conduit. The vacuum source may be configured to apply a suction force that pulls bodily fluids from the porous material and deposits the bodily fluids in the fluid storage container.

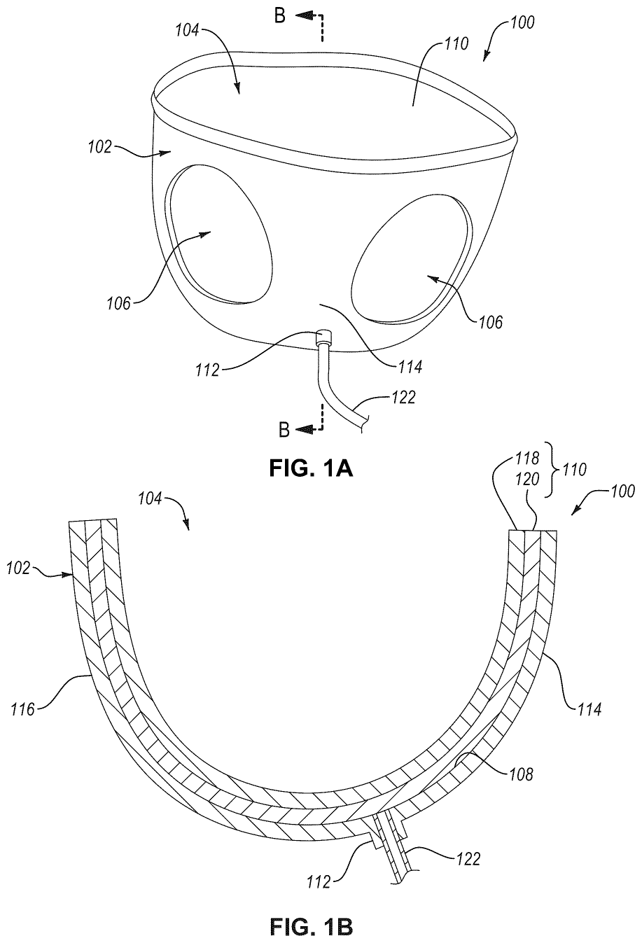

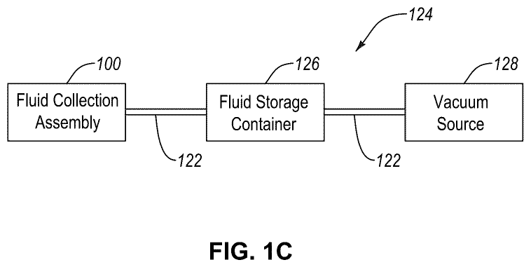

FIG. 1A is an isometric view of a fluid collection assembly 100, according to an embodiment. FIG. 1B is a cross-sectional schematic of the fluid collection assembly 100 taken along plane B-B shown in FIG. 1A, according to an embodiment. The fluid collection assembly 100 includes a fluid impermeable barrier 102. The fluid impermeable barrier 102 at least partially defines a waist opening 104 configured to receive a waist of an individual and two leg openings 106 each configured to receive a leg (e.g., thigh) of an individual. The fluid impermeable barrier 102 includes an interior surface 108. The fluid collection assembly 100 includes at least one porous material 110 positioned adjacent to at least a portion of the interior surface 108 of the fluid impermeable barrier 102. The fluid impermeable barrier 102 also includes a fluid outlet 112 and the fluid collection assembly 100 includes a conduit 122 extending from the fluid outlet 112. The fluid outlet 112 and the conduit 122 allow for the removal of bodily fluids received by the porous material 110.

In an embodiment, the fluid impermeable barrier 102 may exhibit a shape that, when worn, is similar to conventionally used underwear that presses against the waist and the legs. For example, the fluid impermeable barrier 102 may exhibit a shape that is similar to boy shorts underwear, traditional briefs, hipsters underwear, thongs, French cut panties, g-strings, control briefs, seamless underwear, Brazilian briefs, bikini panties, boxer briefs, tanga briefs, booty shorts, high waist brief shapers, high cut briefs, hikini panties, jockstraps, long johns, panty girdle, pant liner, skirtkini, sports underwear, pantaloons, trunks, or any other type of underwear.

The fluid impermeable barrier 102 may be formed of any suitable fluid impermeable material(s), such as a fluid impermeable polymer (e.g., silicone, polypropylene, polyethylene, polyethylene terephthalate, a polycarbonate, etc.), a metal film, natural rubber, another suitable material, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least one or more portions of at least an outer surface of the fluid impermeable barrier 102 may be formed from a soft and/or smooth material, thereby reducing chaffing.

The portions of the fluid impermeable barrier 102 that define the waist opening 104 and the two leg openings 106 are configured to contact the waist and legs of an individual using the fluid collection assembly 100. Contacting the waist and legs of the individual with the fluid impermeable barrier 102 may prevent the formation of passageways between the fluid impermeable barrier 102 and the individual through which bodily fluids may leak. For example, the fluid impermeable barrier 102 may contact the waist and legs of the individual even when the individual moves (e.g., walks, runs) substantially without leaking thereby allowing the fluid collection assembly 100 to be used with high mobility individual. However, the shape and size of the waist and/or legs of the individual may vary.

In an embodiment, at least a portion of the fluid impermeable barrier 102 is flexible (e.g., elastic, stretchable) which allows the fluid impermeable barrier 102 to remain in contact with the waist and legs. The flexibility allows the fluid impermeable barrier 102 to substantially continuously remain in contact with a waist and legs of the individual so long as the size of the waist and legs of the individual are the same size or larger than the waist opening 104 and the legs openings 106, respectively. The fluid impermeable barrier 102 is flexible when at least a portion of the fluid impermeable barrier 102 exhibits a percent elongation that is greater than about 5%, greater than about 10%, greater than about 15%, greater than about 20%, greater than about 25%, greater than about 30%, greater than about 40%, greater than about 50%, greater than about 60%, greater than about 70%, greater than about 80%, greater than about 90%, greater than about 100%, or in ranges of about 5% to about 15%, about 10% to about 20%, about 15% to about 25%, about 20% to about 30%, about 25% to about 40%, about 30% to about 50%, about 40% to about 60%, about 50% to about 70%, about 60% to about 80%, about 70% to about 90%, or about 80% to about 100%. Generally, increasing the percent elongation of the fluid impermeable barrier 102 allows the fluid impermeable barrier 102 to accommodate a larger range of sizes of the waist and legs of the individual. However, increasing the percent elongation of the fluid impermeable barrier 102 may allow for a greater likelihood that the fluid impermeable barrier 102 inadvertently stretches to form passageways between the fluid impermeable barrier 102 and the individual thereby possibly permitting leaks. As such, the percent elongation of the fluid impermeable barrier 102 may be selected based on whether the fluid collection assembly 100 is configured to be used with a wide variety of sizes of individuals or to prevent leaks. Examples of material that exhibit such flexibility include, but are not limited to, elastomeric materials and flexible weaves of hydrophobic fabrics.

In an example, at least substantially all of the fluid impermeable barrier 102 is flexible (e.g., formed from a flexible material). In an example, only one or more select regions of the fluid impermeable barrier 102 are flexible. In such an example, the one or more regions of the fluid impermeable barrier 102 that are flexible generally include at least a portion of the fluid impermeable barrier 102 that defines the waist opening 104 and/or at least a portion of the fluid impermeable barrier 102 that defines the leg openings 106. Thus, the portions of the fluid impermeable barrier 102 that defines the waist opening 104 and/or the leg openings 106 may conform to the waist and/or legs disposed therethrough while the rest of the fluid impermeable barrier 102 may be formed from a non-flexible material (e.g., a material exhibiting better fluid impermeability than the flexible material).

In an embodiment, the fluid impermeable barrier 102 may include one or more ruffles (e.g., pleats) formed therein. The ruffles may be formed in one or more regions of the fluid impermeable barrier 102 that defines the waist opening 104 and/or the leg openings 106. The fluid collection assembly 100 may include a flexible material that extends across the one or more ruffles. The flexible material may cause the ruffles to bunch together when in a relaxed state (e.g., no external tensile forces are applied to the fluid impermeable barrier 102). However, a tensile force may be applied to the flexible material, for example, by disposing a waist and/or legs through the waist opening 104 and/or the leg openings 106, respectively, that is larger than the waist opening 104 and/or the leg openings 106. The ruffles may allow the fluid impermeable barrier 102 to be formed from a greater variety of materials (e.g., inflexible materials, materials exhibiting better fluid impermeability and/or better durability than a flexible material, etc.) than if such portions of the fluid impermeable barrier 102 were formed from a flexible material. However, the ruffles may form one or more fluid passageways between each ruffle through which bodily fluids may leak. The leakage of the bodily fluids may be mitigated by configuring the flexible material to pull each gap between the ruffles substantially closed or to provide an absorbent material adjacent to the ruffles. In an embodiment, the fluid impermeable barrier 102 may include the one or more ruffles when the fluid impermeable barrier 102 is formed from flexible material since the ruffles may allow the size of the waist opening 104 and the leg openings 106 to increase more than if the fluid impermeable barrier 102 did not include the ruffles.

In an embodiment, the fluid impermeable barrier 102 may include a slit formed therein that extends from the waist opening 104 to each of the leg openings 106. The slit formed in the fluid impermeable barrier 102 allows the size of the waist opening 104 and the leg openings 106 to be controllably changed depending on the size of the waist and legs disposed through the waist opening 104 and the legs opening 106, respectively. For example, the waist and legs may be disposed through the waist opening 104 and the leg openings 106, respectively, while opposing portions of the fluid impermeable barrier 102 that form the slits (“opposing portions”) are spaced from each other. The opposing portions may then be brought together until waist opening 104 and the leg openings 106 contact the waist and legs of the individual, respectively. The opposing portions may then be fixedly secured such that the opposing portions do not move. In an example, fixedly securing the opposing portions includes using string, tape, Velcro, etc. to secure at least one of the opposing portions to another portion of the fluid impermeable barrier 102. In an example, the opposing portions are configured to overlap each other before securing the opposing portions which may prevent leaks between the opposing portions.

The fluid impermeable barrier 102 may include a front portion 114 and a back portion 116. The front portion 114 is configured to generally face the same direction as the abdominal region, mons pubis, and area around the urethral opening (e.g., labia folds, portions of the perineum adjacent to the genitals of the individual, etc.) of the individual when the fluid collection assembly 100 is worn. The back portion 116 is configured to generally face the same direction as the back, buttocks, and portion of the perineum adjacent to the anus of the individual when the fluid collection assembly 100 is worn. In an embodiment, the surface area of the front portion 114 of the fluid impermeable barrier 102 is less than the surface area of the back portion 116 since the surface area of the individual covered by the front portion 114 is smaller than the area of the individual covered by the back portion 116. However, in some embodiments, the front and back surfaces 114, 116 have the same surface area and may be indistinguishable from each other. In an embodiment, the front portion 114 may define a bulge near an intersection of the front portion 114 and the back portion 116 that is configured to receive the testicles of the individual when the fluid collection assembly 100 is configured to be used with male anatomy

Referring to FIG. 1B, as previously discussed, the fluid impermeable barrier 102 includes an interior surface 108. The fluid collection assembly 100 includes a porous material 110 disposed adjacent to at least a portion of the interior surface 108. The porous material 110 is disposed adjacent to the portion of the interior surface 108 that is adjacent to the urethral opening of the individual when the fluid collection assembly 100 is used. The portion of the interior surface 108 that is adjacent to the urethral opening of the individual may vary depending on whether the fluid collection assembly 100 is configured to receive bodily fluids from a female urethral opening, male urethral opening, or both. Generally, the portions of the interior surface 108 adjacent to the urethral opening are between the leg openings 106 on the front portion 114 of the fluid impermeable barrier 102. The portions of the interior surface 108 adjacent to a female urethral opening may be closer to an intersection between the front and back portions 114, 116 of the fluid impermeable barrier 102 than portion of the interior surface 108 adjacent to a male urethral opening (e.g., the portions of the interior surface 108 adjacent to the male urethral opening is closer to the waist opening 104). Also, the portion of the interior surface 108 adjacent to a male urethral opening may be greater in size than the portion of the interior surface 108 adjacent to the female urethral opening since the penis may extend outwardly from the individual in a variety of directions and the length of the penis may change.

The porous material 110 may extend from the portion of the interior surface 108 adjacent to the urethral opening to at least the fluid outlet 112. As such, the porous material 110 may channel bodily fluids received thereby towards the fluid outlet 112. The porous material 110 may also extending around the fluid outlet 112 to ensure that the bodily fluids do not inadvertently flow around the fluid outlet 112.

In an embodiment, the porous material 110 may cover other portions of the interior surface 108 in addition to the portions of the interior surface 108 adjacent to the urethral opening, the portions of the interior surface 108 between the portion of the interior surface 108 adjacent to the urethral opening and the fluid outlet 112, and around the fluid outlet 112. For example, the porous material 110 may cover substantially all of the interior surface 108 of the fluid impermeable barrier 102 which may prevent pooling of bodily fluids that where not received by the porous material 110 or left the porous material 110 since pooling of the bodily fluids may damage the skin (e.g., skin degradation), cause discomfort, create unsanitary conditions, and create an odor.

In an embodiment, as previously discussed, the porous material 110 may include a wicking material (e.g., the porous material 110 does not include an adsorbent or absorbent material). The wicking material may be configured to wick any bodily fluids away from the individual and towards the fluid outlet 112. As such, the wicking material may prevent the bodily fluids remaining adjacent to the individual which may cause skin degradation and discomfort, especially since the fluid collection assembly 100 may be configured to be used for prolonged periods of time (e.g., at least 2 hours, at least 3 hours, at least 6 hours, at least 12 hours, at least 18 hours, at least 24 hours, at least 36 hours, about 2 hours to about 6 hours, about 3 hours to about 12 hours, about 6 hours to about 18 hours, about 12 hours to about 24 hours, or about 18 hours to about 36 hours) before being replaced. The permeable properties referred to herein may be wicking, capillary action, diffusion, or other similar properties or processes, and are referred to herein as “permeable” and/or “wicking.” Such “wicking” may not include absorption of fluid into the wicking material. Put another way, substantially no absorption of fluid into the material may take place after the material is exposed to the fluid and removed from the fluid for a time. While no absorption is desired, the term “substantially no absorption” may allow for nominal amounts of absorption of fluid into the porous material 110 (e.g., absorbency), such as less than about 30 wt % of the dry weight of the porous material 110, less than about 20 wt %, less than about 15 wt %, less than about 10 wt %, less than about 7 wt %, less than about 5 wt %, less than about 3 wt %, less than about 2 wt %, less than about 1 wt %, or less than about 0.5 wt % of the dry weight of the porous material 110. The porous material 110 may also wick the fluid generally away from an individual, as discussed in more detail below. It is noted that, in some embodiments, the porous material 110 may include an adsorbent or absorbent material instead of or in addition to the wicking material even though such adsorbent or absorbent materials may not remove the bodily fluids from the individual towards the fluid outlet 112 as much as the wicking material. For example, the adsorbent or absorbent materials may impede leaks when the adsorbent or absorbent materials are at the waist opening 104 and/or the leg openings 106.

The porous material 110 may include one or more of a fluid permeable membrane 118 or a fluid permeable support 120. The fluid permeable membrane 118 may be composed to wick fluid away from the individual to the fluid outlet 112, thereby preventing the fluid from leaking while also removing the bodily fluids from the fluid collection assembly 100. The fluid permeable membrane 118 may include any material that may wick the fluid. For example, the fluid permeable membrane 118 may include fabric, such as a gauze (e.g., a silk, linen, or cotton gauze), another soft fabric, or another smooth fabric. Forming the fluid permeable membrane 118 from gauze, soft fabric, and/or smooth fabric may reduce chaffing caused by the fluid collection assembly 100.

The porous material 110 may include the fluid permeable support 120. The fluid permeable support 120 is configured to support the fluid permeable membrane 118 since the fluid permeable membrane 118 may be formed from a relatively foldable, flimsy, or otherwise easily deformable material. For example, the fluid permeable support 120 may be positioned such that the fluid permeable support 120 is disposed between the fluid permeable membrane 118 and the fluid impermeable barrier 102. As such, the fluid permeable support 120 may support and maintain the position of the fluid permeable membrane 118. The fluid permeable support 120 may include any material that may wick the fluid, such as any of the fluid permeable membrane materials disclosed above. For example, the fluid permeable membrane material(s) may be utilized in a more dense or rigid form than in the fluid permeable membrane 118 when used as the fluid permeable support 120. The fluid permeable support 120 may be formed from any fluid permeable material that is less deformable than the fluid permeable membrane 118. For example, the fluid permeable support 120 may include a porous polymer (e.g., nylon, spun nylon fibers, polyester, polyurethane, polyethylene, polypropylene, etc.) structure or an open cell foam. In some examples, the fluid permeable support 120 may be formed from a natural material, such as cotton, wool, silk, or combinations thereof. In such examples, the material may have a coating to prevent or limit absorption of fluid into the material, such as a water repellent coating. In some examples, the fluid permeable support 120 may be formed from fabric, felt, gauze, or combinations thereof. In some examples, the fluid permeable membrane 118 may be optional. For example, the wicking material may include only the fluid permeable support 120. In some examples, the fluid permeable support 120 may be optionally omitted from the fluid collection assembly 100. For example, the wicking material may only include the fluid permeable membrane 118. The fluid permeable support 120 may have a greater ability to wick fluids than the fluid permeable membrane 118. In some examples, the wicking ability of the fluid permeable support 120 and the fluid permeable membrane 118 may be substantially the same.

In an embodiment, the porous material 110 only includes a single layer, such as one of the fluid permeable membrane 118 or the fluid permeable support 120. In an embodiment, the porous material 110 includes three or more layers, such as an odor reducing layer in addition to the fluid permeable membrane 118 and the fluid permeable support 120.

In an embodiment, at least a portion of the porous material 110 (e.g., one or more of the fluid permeable membrane 118 or, more specifically, the fluid permeable support 120) may be hydrophobic. The porous material 110 may be hydrophobic when the porous material 110 exhibits a contact angle with water (a major constituent of bodily fluids) that is greater than about 90°, such as in ranges of about 90° to about 120°, about 105° to about 135°, about 120° to about 150°, about 135° to about 175°, or about 150° to about 180°. The hydrophobicity of the porous material 110 may limit absorption, adsorption, and solubility of the bodily fluids in the porous material 110 thereby decreasing the amount of bodily fluids held in the porous material 110. In an embodiment, at least a portion of the porous material 110 is hydrophobic or hydrophilic. In an embodiment, the fluid permeable support 120 is more hydrophobic (e.g., exhibits a larger contact angle with water) than the fluid permeable membrane 118. The lower hydrophobicity of the fluid permeable membrane 118 may help the porous material 110 receive the bodily fluids from the urethral opening while the hydrophobicity of the fluid permeable support 120 limits the bodily fluids that are retained in the porous material 110.

As previously discussed, the fluid impermeable barrier 102 defines the fluid outlet 112. Generally, the fluid outlet 112 is at or near a gravimetric low point of the fluid collection assembly 100. Locating the fluid outlet 112 at or near a location expected to be the gravimetrically low point of the fluid collection assembly 100 when worn by an individual enables the conduit 122 to receive more of the bodily fluids than if fluid outlet 112 was located elsewhere and reduce the likelihood of pooling (e.g., pooling of the bodily fluids may cause microbe growth and foul odors). For instance, the bodily fluids in the fluid permeable membrane 118 and the fluid permeable support 120 may flow in any direction due to capillary forces. However, the bodily fluids may exhibit a preference to flow in the direction of gravity, especially when at least a portion of the fluid permeable membrane 118 and/or the fluid permeable support 120 is saturated with the bodily fluids. Accordingly, fluid outlet 112 may be located in the fluid collection assembly 100 in a position expected to be the gravimetrically low point in the fluid collection assembly 100 when worn by an individual. The gravimetric low point of the fluid collection assembly 100 may vary depending on the position of the individual. For example, the gravimetric low point of the fluid collection assembly 100 may be at or near the intersection of the front and back portions 114, 116 of the fluid impermeable barrier 102 (e.g., at or near a portion of the fluid impermeable barrier adjacent to a perineum or genitalia of the individual) fluid outlet when the individual is standing, on the back portion 116 near the perineum and/or anus of the individual when the individual is sitting, on the back portion 116 near the anus of the individual or between the anus and waist opening 104 when the individual is laying down on the individual's back, or on the front portion 114 when the individual is laying on the individual's stomach.

The fluid outlet 112 is sized to receive the conduit 122. For example, the fluid outlet 112 may be sized and shaped to form an at least substantially fluid tight seal against the conduit 122 or the at least one tube thereby substantially preventing the bodily fluids from leaking from the fluid collection assembly 100.

The conduit 122 may be used to remove fluid form the fluid collection assembly 100. The conduit 122 (e.g., a tube) includes an inlet and an outlet positioned downstream from the inlet. The outlet may be operably coupled to a suction source, such as a vacuum pump for withdrawing fluid from the fluid collection assembly 100 through the conduit 122. For example, the conduit 122 may extend into the fluid impermeable barrier 102. The conduit 122 fluidly couples the fluid collection assembly 100 with the fluid storage container (not shown) or the vacuum source (not shown).

The conduit 122 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some examples, the conduit 122 may include silicon or latex. In some examples, the conduit 122 may include one or more portions that are resilient, such as to by having one or more of a diameter or wall thickness that allows the conduit to be flexible.

In an example, the conduit 122 is configured to be at least insertable to be adjacent to the porous material 110. In such an example, the conduit 122 may include one or more markers (not shown) on an exterior thereof that are located to facilitate insertion of the conduit 122 into the fluid outlet 112. For example, the conduit 122 may include one or more markings thereon that are configured to prevent over or under insertion of the conduit 122. In another example, the conduit 122 may include one or more markings thereon that are configured to facilitate correct rotation of the conduit 122. The one or more markings may include a line, a dot, a sticker, or any other suitable marking.

In an embodiment, the fluid collection assembly 100 is configured to be washable. The fluid collection assembly 100 is washable when the components of the fluid collection assembly 100 (e.g., the fluid impermeable barrier 102 and the porous material 110) may be washed substantially without damaging the components of the fluid collection assembly 100. For example, the components of the fluid collection assembly 100 may be configured to not be softened or dissolved in water and may remain attached to each other during the tumbling and spinning cycles of the washing machine. Allowing the fluid collection assembly 100 to be washable allows the fluid collection assembly 100 to be reused which makes using the fluid collection assembly 100 more economical to use and reduces waste. Further, the fluid collection assembly 100 may be machine dryable. The fluid collection assembly 100 is machine dryable when the fluid collection assembly 100 may be spun in the clothes drier at temperature of about 135° C. substantially without damage. In an embodiment, the fluid collection assembly 100 is configured for single use. While a fluid collection assembly 100 configured for single use may increase waste, it allows the fluid collection assembly 100 to be manufactured from a wider range of materials.

FIG. 1C is a block diagram of a fluid collection system 124 for fluid collection, according to an embodiment. The fluid collection system 124 includes a fluid collection assembly 100, a fluid storage container 126, and a vacuum source 128. The fluid collection assembly 100, the fluid storage container 126, and the vacuum source 128 may be fluidly coupled to each other via one or more conduits 122. For example, fluid collection assembly 100 may be operably coupled to one or more of the fluid storage container 126 or the vacuum source 128 via the conduit 122. Bodily fluids collected in the fluid collection assembly 100 may be removed from the fluid collection assembly 100 via the conduit 122. Suction force may be introduced into the fluid collection assembly 100 via the inlet of the conduit 122 responsive to suction (e.g., vacuum) force applied at the outlet of the conduit 122.

The suction force may be applied to the outlet of the conduit 122 by the vacuum source 128 either directly or indirectly. The suction force may be applied indirectly via the fluid storage container 126. For example, the outlet of the conduit 122 may be disposed within the fluid storage container 126 and an additional conduit 122 may extend from the fluid storage container 126 to the vacuum source 128. Accordingly, the vacuum source 128 may apply suction to the fluid collection assembly 100 via the fluid storage container 126. The suction force may be applied directly via the vacuum source 128. For example, the outlet of the conduit 122 may be disposed within the vacuum source 128. An additional conduit 122 may extend from the vacuum source 128 to a point outside of the fluid collection assembly 100, such as to the fluid storage container 126. In such examples, the vacuum source 128 may be disposed between the fluid collection assembly 100 and the fluid storage container 126.

The fluid storage container 126 is sized and shaped to retain a fluid therein. The fluid storage container 126 may include a bag (e.g., drainage bag), a bottle or cup (e.g., collection jar), or any other enclosed container for storing bodily fluids such as urine. In some examples, the conduit 122 may extend from the fluid collection assembly 100 and attach to the fluid storage container 126 at a first point therein. An additional conduit 122 may attach to the fluid storage container 126 at a second point thereon and may extend and attach to the vacuum source 128. Accordingly, a vacuum (e.g., suction) may be drawn through the fluid collection assembly 100 via the fluid storage container 126. Bodily fluids, such as urine, may be drained from the fluid collection assembly 100 using the vacuum source 128.

The vacuum source 128 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 128 may provide a vacuum or suction to remove fluid from the fluid collection assembly 100. In an example, the vacuum source 128 may be powered by one or more of a power cord (e.g., connected to a power socket), one or more batteries, or even manual power (e.g., a hand operated vacuum pump). In an example, the vacuum source 128 may be sized and shaped to fit outside of, on, or within the fluid collection assembly 100. In an example, the vacuum source 128 may include one or more miniaturized pumps or one or more micro pumps. The vacuum source 128 disclosed herein may include one or more of a switch, a button, a plug, a remote, or any other device suitable to activate the vacuum source 128.

Referring back to FIGS. 1A and 1B, the fluid collection assembly 100 includes the porous material 110 adjacent to the interior surface 108 of the fluid impermeable barrier 102 such that there are no substantially unoccupied spaces between the porous material 110 and the interior surface 108. The lack of an occupied spaced between the interior surface 108 and the porous material 110 may inhibit pooling of the bodily fluids between the interior surface 108 and the porous material 110. Further, as will be discussed in more detail below, the lack of an unoccupied space between the interior surface 108 and the porous material 110 may allow the fluid collection assembly 100 to be worn more discretely and/or increase the volume of the porous material 110. In an example, at least a significant portion of a surface of the porous material 110 that faces the interior surface 108 of the fluid impermeable barrier 102 and the interior surface 108 are attached together to inhibit the porous material 110 becoming spaced from the interior surface 108. In an example, the porous material 110 corresponds to the interior surface 108 such that the porous material 110 rests against the interior surface 108 substantially without forming any unoccupied spaces therebetween when no external forces pull the porous material 110 and the interior surface 108 apart.

However, the fluid collection assemblies disclosed herein may include at least one substantially unoccupied spaced between the interior surface of the fluid impermeable barrier and the porous material. The substantially unoccupied space is referred to herein as a fluid reservoir. In an example, the fluid reservoir may increase the volume of the bodily fluids that may be temporarily stored by the fluid collection assembly. For instance, the rate of bodily fluids introduced into the fluid collection assembly when an individual urinates may be greater than the rate at which the bodily fluids are removed from the fluid collection assembly. The substantially unoccupied space provides a location for the bodily fluids to pool while waiting to be removed from the fluid collection assembly. The substantially unoccupied space also minimizes oversaturation of the porous material which may cause the fluid collection assembly to leak or cause the bodily fluids to remain in contact with the individual thereby causing skin degradation and discomfort.

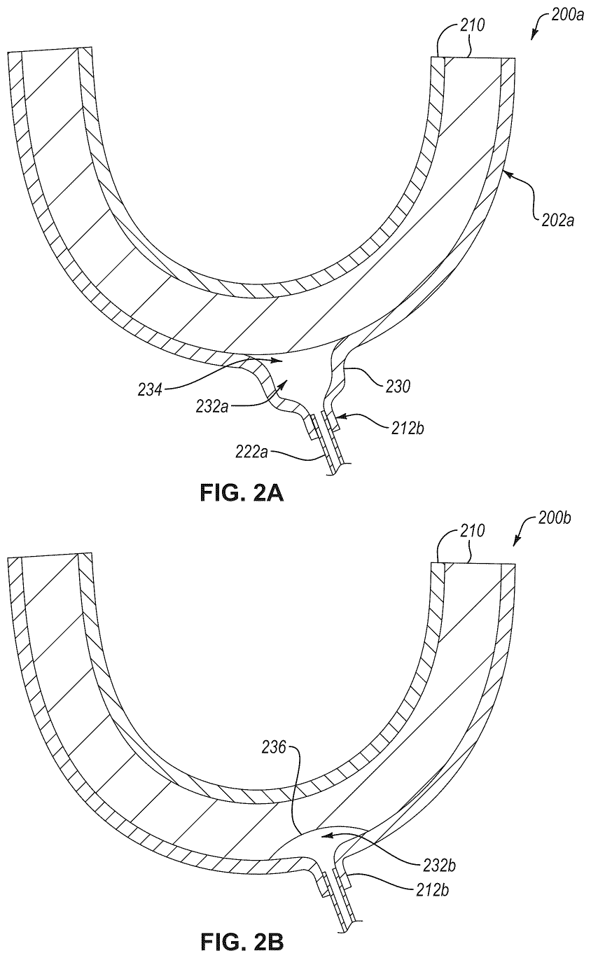

FIGS. 2A and 2B are cross-sectional views of fluid collection assemblies that include fluid reservoirs, according to different embodiments. Except as otherwise disclosed herein, the fluid collection assemblies of FIGS. 2A and 2B are substantially similar to any of the fluid collection assemblies disclosed herein. For example, the fluid collection assemblies of FIGS. 2A and 2B may include a fluid impermeable barrier defining a waist opening, two leg openings, and a fluid outlet. The fluid collection assemblies of FIGS. 2A and 2B may also include at least one porous material adjacent to a portion of an interior surface of the fluid collection assemblies. It is noted that the fluid collection assemblies illustrated in FIGS. 2A and 2B may be used in any of the fluid collection systems disclosed herein.

Referring to FIG. 2A, the fluid impermeable barrier 202a of the fluid collection assembly 200a includes a bulge 230 extending outwardly (e.g., away from the porous material 210a) from the rest of the fluid impermeable barrier 202a. The porous material 210a does not exhibit a shape that corresponds to the bulge 230 such that the bulge 230 defines a fluid reservoir 232a (e.g., substantially unoccupied space) that may temporarily store bodily fluids therein. For example, the bulge 230 may define an inlet 234. The porous material 210a may extend across the inlet 234 such that the porous material 210a substantially does not extend into the space defined by the bulge 230 or the porous material 210a may only extend partially into the space defined by the bulge 230. It is noted that the bulge 230 may make wearing the fluid collection assembly 200a less discrete since the bulge 230 is more likely to be noticeable through outerwear (e.g., the bulge 230 presses against pants or shorts worn by the individual) than if the fluid collection assembly 200a did not include the bulge 230.

In an embodiment, the fluid outlet 212b extends from, is formed by, or is otherwise in fluid communication with the bulge 230. As such, the fluid outlet 212b may remove bodily 20) fluids temporarily stored in the bulge 230 thereby substantially preventing the bodily fluids from pooling in the fluid reservoir 232a. The bulge 230 may be located at or near the gravimetric low point of the fluid collection assembly 200a and the fluid outlet 212b may be located at or near the gravimetric low point of the bulge 230. For example, as previously discussed, bodily fluids received by the porous material 210a are preferentially drawn to the gravimetric low point of the fluid collection assembly 200. Thus, the bodily fluids may be preferentially drawn to and deposited in the bulge 230. Similarly, the bodily fluids deposited in the bulge 230 may be located at the gravimetric low point of the bulge 230. Locating the fluid outlet 212b at or near the gravimetric low point of the bulge 230 allows the fluid outlet 212b to remove bodily fluids from the fluid reservoir 232a. If the fluid outlet 212b is spaced from the gravimetric low point of the bulge 230 and the conduit 222a does not extend into the fluid reservoir 232a, the fluid outlet 212b may only remove bodily fluids from the bulge 230 when the quantity of the bodily fluids in the bulge 230 is sufficient to reach the fluid outlet 212b since the fluid reservoir 232a is substantially unoccupied.

Referring to FIG. 2B, the porous material 210b of the fluid collection assembly 200b defines a cutout 236 that does not conform to the interior surface 208 of the fluid impermeable barrier 202b. Thus, the cutout 236 forms a fluid reservoir 232b. However, the cutout 236 decreases the volume of the porous material 210b that may receive and temporarily retain the bodily fluids therein.

In an embodiment, the cutout 236 is adjacent to and/or surrounds the fluid outlet 212b. As such, the fluid outlet 212b may remove bodily fluids temporarily stored in the fluid reservoir 232b thereby substantially preventing the bodily fluids from pooling in the fluid reservoir 232b. The cutout 236 and the fluid outlet 212b may be located at or near the gravimetric low point of the fluid collection assembly 200b. For example, as previously discussed, bodily fluids received by the porous material 210b are preferentially drawn to the gravimetric low point of the fluid collection assembly 200b. Thus, the bodily fluids may be preferentially drawn to and deposited in the fluid reservoir 232b and allows the fluid outlet 212b to remove bodily fluids from the fluid reservoir 232b.

As previously discussed, the gravimetric low point of the fluid collection assemblies disclosed herein depend on the position of the individual. For example, the gravimetric low point of the fluid collection assembly may be at or near the intersection of the front and back portions of the fluid impermeable barrier when the individual is standing, on the back portion near the perineum and/or anus of the individual when the individual is sitting, on the back portion near the anus of the individual or between the anus and waist opening when the individual is laying down on the individual's back, or on the front portion when the individual is laying on the individual's stomach. Also, as previously discussed, the fluid collection assemblies disclosed herein allow the individual to move (e.g., switch from a sitting to a standing position). As such, moving the individual may cause the gravimetric low point of the fluid collection assembly to change. Fluid collection assemblies that only include a single fluid outlet may require the individual to assume a certain position when the individual discharges bodily fluids (e.g., urinates) or when the individual senses bodily fluids in the fluid collection assembly to ensure that fluid outlet is at or near the gravimetric low point. Alternatively, the fluid collection assemblies may include a plurality of fluid outlets to facilitate removal of the bodily fluids from the fluid collection assembly when the individual assumes a plurality of positions.

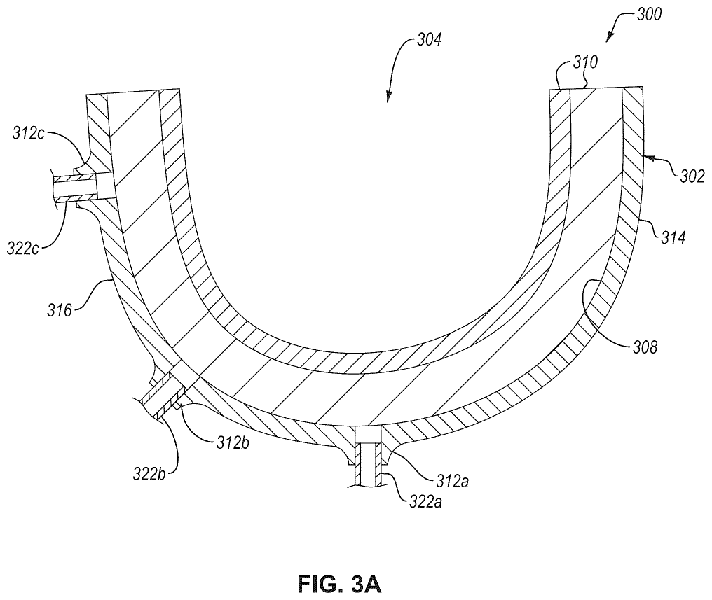

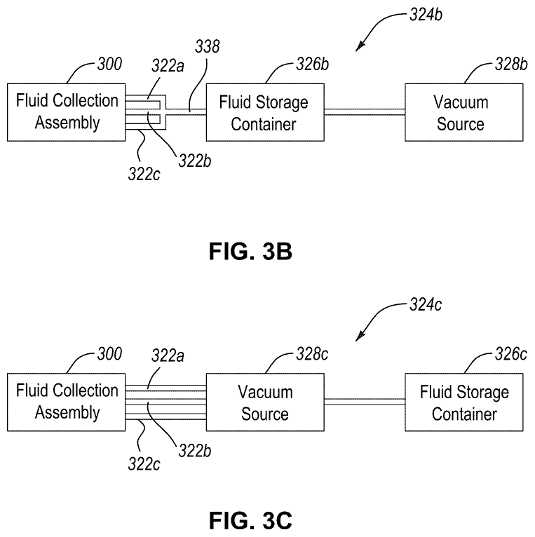

FIG. 3A is a cross-sectional schematic of a fluid collection assembly 300 that includes a plurality of fluid outlets, according to an embodiment. Except as otherwise disclosed herein, the fluid collection assembly 300 is the same or substantially similar to any of the fluid collection assemblies disclosed herein. For example, the fluid collection assembly 300 may include a fluid impermeable barrier 302 that defines a waist opening 304 and two leg openings (not shown). The fluid collection assembly 300 also includes at least one porous material 310 adjacent to at least a portion of an interior surface 308 of the fluid impermeable barrier 302. It is noted that the fluid collection assembly 300 may be used in any of the fluid collection systems disclosed herein.

The fluid impermeable barrier 302 defines a plurality of fluid outlets. For example, in the illustrated embodiment, the fluid impermeable barrier 302 defines a first fluid outlet 312a, a second fluid outlet 312b, and a third fluid outlet 312c. The first fluid outlet 312a may be configured to be at or near a gravimetric low point of the fluid collection assembly 300 when an individual using the fluid collection assembly 300 is standing. As such, the first fluid outlet 312a may be at or near an intersection of the front and back portions 314, 316 of the fluid impermeable barrier 302. The second fluid outlet 312b may be configured to be at or near a gravimetric low point of the fluid collection assembly 300 when an individual using the fluid collection assembly 300 is sitting. As such, the second fluid outlet 312b may be on the back portion 316 of the fluid impermeable barrier 302 at or near the perineum and/or anus of the individual. The third fluid outlet 312c may be configured to be at or near a gravimetric low point of the fluid collection assembly 300 when an individual using the fluid collection assembly 300 is lying on the individual's back. As such, the third fluid outlet 312c may be on the back portion 316 of the fluid impermeable barrier 302 near the anus of the individual or between the anus and waist opening 304. The fluid collection assembly 300 may include a first conduit 322a, a second conduit 322b, and a third conduit 322c coupled to the first fluid outlet 312a, the second fluid outlet 312b, and the third fluid outlet 312c. As such, the fluid collection assembly 300 may have substantially all the bodily fluid removed therefrom regardless if the individual using the fluid collection assembly 300 is standing, sitting, or lying on the individual's back.

It is noted that the fluid impermeable barrier 302 may define more (e.g., four, five, etc.) or less (e.g., two) than the three fluid outlets illustrated in FIG. 3A. Further, the fluid impermeable barrier 302 may define fluid outlets at locations other than the locations illustrated in FIG. 3A. In an example, the fluid impermeable barrier 302 may define fluid outlets that are located on the front portion 314 of the fluid impermeable barrier 302 closer to the waist opening 304 than the first fluid outlet 312a to facilitate the removal of bodily fluids when the individual is laying on the individual's stomach. In an example, the fluid impermeable barrier 302 may define fluid outlet that are located closer to one leg opening than another leg opening which may facilitate removal of bodily fluids when the individual is laying on the individual's side. In an example, the fluid impermeable barrier 302 may define fluid outlets that are located between the fluid outlets illustrated in FIG. 3A or otherwise disclosed herein. In such an example, the fluid outlets may facilitate the removal of bodily fluids when the individual is in an intermediate position, such as a slouching.

FIG. 3B is a block diagram of a fluid collection system 324b that includes the fluid collection assembly 300 illustrated in FIG. 3A, according to an embodiment. Except as otherwise disclosed herein, the fluid collection system 324b may be the same or substantially similar to any of the systems disclosed herein. For example, the fluid collection system 324b may include a fluid storage container 326b and a vacuum source 326b that are in fluid communication with each other and with the fluid collection assembly 300 via one or more conduits.

As previously discussed, the fluid collection assembly 300 includes a first conduit 322a, a second conduit 322b, and a third conduit 322c. The first conduit 322a, the second conduit 322b, and the third conduit 322c may extend away from the fluid collection assembly 300 to a common conduit 338. The common conduit 338 may extend from the first conduit 322a, the second conduit 322b, and the third conduit 322c to the fluid storage container 326b, as shown, or to the vacuum source 328b. Any suction force applied to the common conduit 338 is also applied to the first conduit 322a, the second conduit 322b, and the third conduit 322c. It is noted that the fluid collection system 324b may include more or fewer conduits than the first conduit 322a, the second conduit 322b, and the third conduit 322c, depending on the number of fluid outlets formed in the fluid collection assembly 300.

FIG. 3C is a block diagram of a fluid collection system 324c that includes the fluid collection assembly 300 illustrated in FIG. 3A, according to an embodiment. Except as otherwise disclosed herein, the fluid collection system 324c may be the same or substantially similar to any of the systems disclosed herein. For example, the fluid collection system 324c may include a fluid storage container 326c and a vacuum source 328c that are in fluid communication with each other and with the fluid collection assembly 300 via one or more conduits.

As previously discussed, the fluid collection assembly 300 includes a first conduit 322a, a second conduit 322b, and a third conduit 322c. The first conduit 322a, the second conduit 322b, and the third conduit 322c may extend away from the fluid collection assembly 300. The first conduit 322a, the second conduit 322b, and the third conduit 322c may not intersection with each other but, instead may be in individually fluidly coupled to the vacuum source 328c (as shown) or to the fluid storage container 326c. As such, the vacuum source 328c may apply a suction force to one or more of the first conduit 322a, the second conduit 322b, and the third conduit 322c. It is noted that the fluid collection system 324c may include more or fewer conduits than the first conduit 322a, the second conduit 322b, and the third conduit 322c, depending on the number of fluid outlets formed in the fluid collection assembly 300.

With regards to the fluid collection system 324b illustrated in FIG. 3B or the fluid collection system 324c illustrated in FIGS. 3C, a suction force provided to by the vacuum source of the system may be applied to all of the conduits coupled to the fluid outlets simultaneously (e.g., applied to the first conduit 322a, the second conduit 322b, and the third conduit 322c simultaneously) or may be applied selectively to only some of the conduits coupled to the fluid outlet (e.g., applied selectively to one or two of the first conduit 322a, the second conduit 322b, and the third conduit 322c). Applying the suction force to all of the conduits coupled to the fluid outlets simultaneously may allow for the removal of bodily fluids from the fluid collection assembly 300 using each of the fluid outlets. For example, applying the suction force to all of the conduits may allow for removal of the bodily fluids at or near the gravimetric low point of the fluid collection assembly and for the removal of the bodily fluids that are spaced from the gravimetric low point of the fluid collection assembly since, while the bodily fluids flow preferentially towards the gravimetric low point, some of the bodily fluids may flow away from the gravimetric low point due to the wicking. However, the suction force applied to the fluid outlet at the gravimetric low point may be less than if the suction force was only applied to the fluid outlet at the gravimetric low point thereby decreasing the rate at which the bodily fluids are removed from the fluid outlet at the gravimetric low point. Also, the suction force may preferentially remove air from the fluid collection assembly than liquids. Thus, applying the suction force to each of the fluid outlets may reduce the rate at which bodily fluids are removed from the fluid collection assembly when one of the fluid outlets is exposed to air.

Alternatively, the fluid collection systems 324b and/or 324c may be configured to apply the suction force selectively to some of the conduits coupled to the fluid outlets of the fluid collection assembly. Selectively applying the suction force may mitigate issues caused by the suction force preferentially pulling air instead of bodily fluids and increase the suction force applied to the fluid outlet that is exposed to bodily fluids. In an example, the first conduit 322a, the second conduit 322b, and the third conduit 322c of the fluid collection system 324b illustrated in FIG. 3B may include valves that selectively open (e.g., allow a flow of the suction force through the conduit) or close (e.g., restriction a flow the suction force through the conduit). In other words, the valves may be used to apply the suction force to selected one(s) of the conduits coupled to the fluid outlets of the fluid collection assembly. In an example, the vacuum source 328c of the fluid collection system 324c illustrated in FIG. 3C may be configured to selectively apply the suction force to selected one(s) of the conduits coupled to the fluid outlets of the fluid collection assembly.

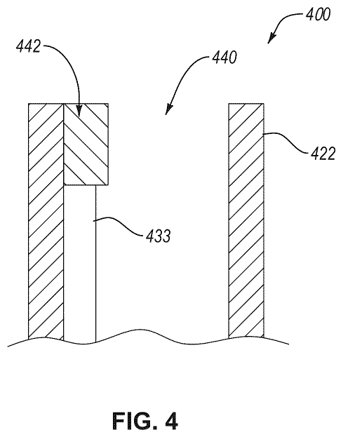

FIG. 4 is a cross-sectional schematic of a portion of a fluid collection assembly 400, according to an embodiment. Except as otherwise disclosed herein, the fluid collection assembly 400 may be the same or substantially similar to any of the fluid collection assemblies disclosed herein. For example, the fluid collection assembly 400 includes a conduit 422. The conduit 422 includes an inlet 440 that is positioned within or proximate to (e.g., inside the fluid reservoir) to a fluid outlet (not shown). The inlet 440 allows the conduit 422 to receive bodily fluids.

The fluid collection assembly 400 includes a sensor 442 that is configured to detect a presence of moisture (e.g., moisture caused by the presence of bodily fluids). The sensor 442 may be configured to detect the presence of moisture directly or indirectly (e.g., detect the absence of air). The sensor 442 may include any sensor that may detect the presence of moisture, such as one or more of an electrochemical gas sensor, a humistor, a hygrometer, a flow meter, a pH sensor, or two or more electrodes.

In an embodiment, the sensor 442 is disposed in the conduit 422. In such an embodiment, the sensor 442 may determine whether the conduit 422 is removing air or bodily fluids from the rest of the fluid collection assembly 400. In an embodiment, the sensor 442 is disposed outside of the conduit 422, such as proximate to the conduit 422 and at least one of in a fluid reservoir, adjacent to the fluid impermeable barrier, or within the porous material. In such an embodiment, the sensor 442 may detect the presence of the bodily fluids even when the bodily fluids are not flowing through the conduit 422 (e.g., when a suction force is not applied to the conduit 422). In an embodiment, the sensor 442 is disposed at the inlet 440 of the conduit 422 which allows the sensor 442 to detect when the bodily fluids are flowing through the conduit 422 and the presence of the bodily fluids even when the bodily fluids are not flowing through the conduit 422.

The sensor 442 may be communicably coupled to one or more components of a fluid collection system (not shown) via a wired connection 443 (as shown) or a wireless connection. For example, a fluid collection system that include the fluid collection assembly 400 may include a controller having control electrical circuitry (not shown). The controller may include non-transitory memory storing one or more operational instructions and at least one processor configured to execute the operational instructions. The controller may be communicably coupled to the sensor 442 and one or more components of the fluid collection system (e.g., the one or more valves or the vacuum source). The controller may receive one or more signals from the sensor 442. The signals may indicate whether the sensor 442 detected or did not detect bodily fluids. The controller may control the operation of the valves and/or the vacuum source response to the signals from the sensor 442. For example, responsive to instructions from the controller, the valves or vacuum source may be configured to selectively apply a suction force to the conduit 422 depending on whether the sensor 442 detected or did not detect the bodily fluids. In an embodiment, the fluid collection assembly 400 may include a plurality of conduits, similar 25 to the embodiments discussed with regards to FIGS. 3A-3C. In such an embodiment, the controller may direct the valves and/or vacuum source to apply the suction force only to the conduit(s) associated with sensor(s) 442 that detected bodily fluids to increase the suction force applied to such conduits and to minimize air that is pulled through the conduit(s). In an embodiment, the control electrical circuitry may direct the vacuum source to only apply a suction force to the conduit 422 when the sensor 442 detects the presence of the bodily fluids. Applying the suction force only when the sensor 442 detects the presence of the bodily fluids may reduce the time periods during which the vacuum source generates noise thereby allowing the fluid collection assembly 400 to be used more discretely.

It is noted that the suction force applied to the conduit 422 may be controlled in other ways, including with or without the sensor 442. In an example, the valves may include actuators that may be manipulated by the individual. Manipulating the valves may switch the valves between an open and closed state. In an example, the vacuum source may include one or more actuators (e.g., buttons) and manipulating the actuator may cause the vacuum source to at least one of selectively apply the suction source to one or more of the conduits or turn on and off the vacuum source.

As previously discussed, the fluid collection systems disclosed herein may be used with any suitable vacuum source. In an embodiment, the fluid collection systems disclosed herein include a vacuum source that include an in-the-wall vacuum source (e.g., an access port, such as inlet, of the in-the-wall vacuum source in provided at a wall) or a plugged-in vacuum source. In such an embodiment, the distance that an individual wearing a fluid collection assembly may move is limited by the length of the conduit that connects the individual to the in-the-wall vacuum source or the plugged-in vacuum source. As such, the mobility of the individual is limited even though the fluid collection assembly worn by the individual allows for significant movement.

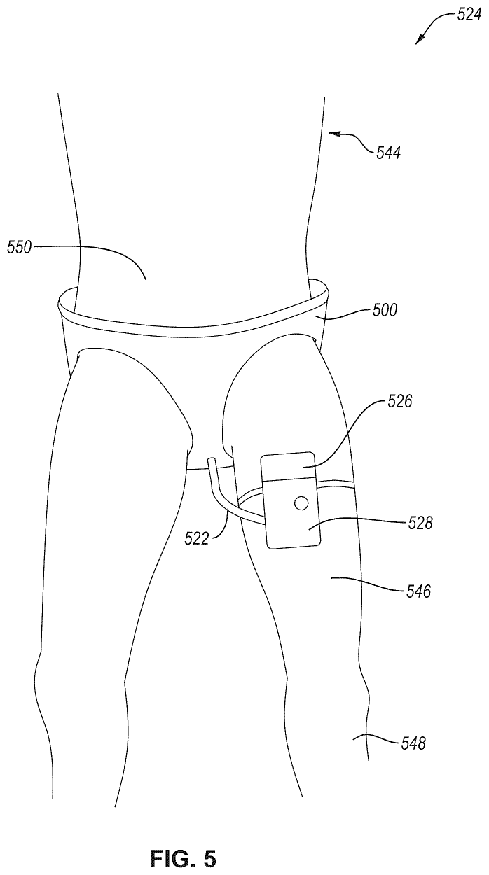

In an embodiment, the fluid collection systems disclosed herein may include a portable vacuum source which allows the individual wearing any of the fluid collection assemblies disclosed herein significant movement. FIG. 5 is an isometric view of a fluid collection system 524 that includes a portable vacuum source 528 on an individual 544, according to an embodiment. Except as otherwise disclosed herein, the fluid collection system 524 may be the same or substantially similar to any of the fluid collection systems disclosed herein. For example, the fluid collection system 524 may include a fluid collection assembly 500. The fluid collection assembly 500 may include any of the fluid collection assemblies disclosed herein. The fluid collection system 524 also includes a fluid storage container 526 and the vacuum source 528. The fluid storage container 526 and the vacuum source 528 may be in fluid communication with the fluid collection assembly 500 via at least one conduit 522.

The fluid storage container 526 and the vacuum source 528 are configured to be worn by the individual 544. For example, in the illustrated embodiment, the fluid storage container 526 and the vacuum source 528 are configured to be attached to the thigh 546 of the individual 544. In such an example, the fluid storage container 526 and the vacuum source 528 exhibit a size and shape that allows the fluid storage container 526 and the vacuum source 528 to be positioned on the thigh 546 of the individual 544. For instance, the fluid storage container 526 and the vacuum source 528 collectively may exhibit a length measured along a longitudinal axis of the thigh 546 that is less than a 25 cm (e.g., less than 20 cm, less than 15 cm, or less than 10 cm), a width that is less than 20 cm (e.g., less than 15 cm, less than 10 cm, less than 7.5 cm, or less than 5 cm), and a thickness that is less than 7.5 cm (e.g., less than 5 cm, less than 4 cm, less than 3 cm, or less than 2 cm). Such sizes may allow the fluid storage container 526 and the vacuum source 528 to be worn discretely on the thigh 546 of the individual 544. Further, the fluid storage container 526 and the vacuum source 528 may include a concave surface that is configured to mate with the thigh 546 of the individual 544 since such a concave surface may make wearing the fluid storage container 526 and the vacuum source 528 more comfortable. The fluid storage container 526 and the vacuum source 528 may be attached to the thigh 546 using any suitable attachment mechanism, such as with a strap or an adhesive.

It is noted that the fluid storage container 526 and the vacuum source 528 may be configured to be worn on a different area of the individual 544. In an example, the fluid storage container 526 and the vacuum source 528 may be configured to be worn on the calf 548 or abdominal region 550 of the individual. In such an example, the fluid storage container 526 and the vacuum source 528 may exhibit any of the size or shapes discussed above. In an example, the fluid storage container 526 and the vacuum source 528 may be configured to be disposed in a pocket or attached to a belt.

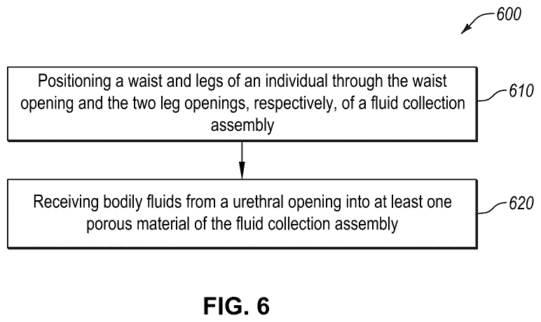

FIG. 6 is a flow diagram of a method 600 to collect fluid, according to an embodiment. The method 600 of collecting fluid may utilize use any of the fluid collection assemblies and/or fluid collection systems disclosed herein. The method 600 may include act 610, which recites “positioning a waist and legs of an individual through the waist opening and the two leg openings, respectively, of a fluid collection assembly.” Act 610 may be followed by act 620, which recites “receiving bodily fluids from a urethral opening into at least one porous material of the fluid collection assembly.”

Acts 610, 620 of the method 600 are for illustrative purposes. For example, the act 610, 620 of the method 600 may be performed in different orders, split into multiple acts, modified, supplemented, or combined. In an example, one or more of the acts 610, 620 of the method 600 may be omitted from the method 600. Any of the acts 610 or 620 may include using any of the fluid collection assemblies or systems disclosed herein.

Act 610 recites “positioning a waist and legs of an individual through the waist opening and the two leg openings, respectively, of a fluid collection assembly.” The act 610 of may include utilizing any of the fluid collection assemblies or systems disclosed herein. In some examples, act 610 may include positioning the fluid collection assembly such that the porous material of the fluid collection assembly abuts or is positioned proximate to the female or male urethral opening.

Act 620 recites “receiving bodily fluids from a urethral opening into at least one porous material of the fluid collection assembly.” In some examples, receiving bodily fluids from a urethral opening (e.g., female or male urethral opening) into porous material of the fluid collection assembly may include wicking the bodily fluids using the at least one porous material, such as via a fluid permeable membrane and a fluid permeable support. Receiving bodily fluids from the urethral opening may include flowing the bodily fluids towards a fluid outlet For instance, receiving fluid from the urethral opening may include flowing the bodily fluids to a substantially unoccupied portion (e.g., a fluid reservoir), to a gravimetrically low point of the fluid collection assembly, etc., such as via gravity, wicking, or suction force.

The method 600 may include applying suction with a vacuum source effective to suction the bodily fluids from the fluid collection assembly via a conduit disposed therein and fluidly coupled to the vacuum source may include using any of the vacuum sources disclosed herein. Applying suction with a vacuum source may include activating the vacuum source (e.g., suction device) in fluid communication with the conduit in the fluid collection assembly. In some examples, activating the vacuum source in fluid communication with the conduit in the fluid collection assembly may include supplying power to the vacuum source by one or more of flipping an on/off switch, pressing a button, plugging the vacuum source into a power outlet, putting batteries into the vacuum source, etc. In some examples, the vacuum source may include a hand operated vacuum pump and applying suction with a vacuum source may include manually operating the hand operated vacuum pump effective to suction the bodily fluids from the fluid collection assembly via the conduit disposed therein that is fluidly coupled to the vacuum source.

In some examples, applying suction with a vacuum source effective to suction the bodily fluids via a conduit disposed therein and fluidly coupled to the vacuum source may be effective to remove at least some bodily fluids (e.g., urine) from the fluid collection assembly. In some examples, applying suction with a vacuum source effective to suction the bodily fluids from the fluid collection assembly via a conduit disposed therein and fluidly coupled to the vacuum source may be effective to transfer at least some of the bodily fluids to a fluid storage container (e.g., a bottle or bag), such as from one or more of a reservoir, fluid permeable support, or fluid permeable membrane.

In some examples, applying suction with a vacuum source effective to suction the bodily fluids via a conduit disposed therein and fluidly coupled to the vacuum source may include detecting moisture in the porous material (e.g., via one or more moisture sensors) and responsive thereto, activating the vacuum source to provide suction in the fluid collection assembly. The control of the vacuum source responsive to the signals indicating that moisture or a level thereof is present in the fluid collection assemly may be automatic, such as via a controller (e.g., computer programmed to perform the operation), or may merely provide an indication that a level of moisture is present that may necessitate removal of fluid from the fluid collection assembly. In the latter case, a user may receive the indication (e.g., from the 20) controller) and activate the vacuum pump manually.

In an example, the method 600 may include collecting the bodily fluids that are removed from the fluid collection assembly, such as into a fluid storage container that is spaced from the fluid collection assembly and fluidly coupled to the conduit. The fluid storage container may include any of the fluid storage containers disclosed herein.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean ±10%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. (canceled)

2. A fluid collection assembly, comprising:
a fluid impermeable barrier at least partially defining:
an interior region;
at least one opening extending through the fluid impermeable barrier to the interior region; and
at least one fluid outlet;
at least one porous material disposed in the interior region generally conforming to at least a portion of the fluid impermeable barrier;
at least one conduit in fluid communication with the interior region via the at least one fluid outlet; and
at least one sensor configured to detect presence of moisture within the fluid collection assembly, the at least one sensor positioned within the at least one conduit, the at least one fluid outlet, or in a portion of the interior region proximate to the at least one fluid outlet.

3. The fluid collection assembly of claim 2, wherein the at least one sensor is positioned within the at least one conduit.

4. The fluid collection assembly of claim 3, wherein the at least one fluid outlet includes a plurality of fluid outlets, the at least one conduit includes a plurality of conduits, and the at least one sensor includes a plurality of sensors;
wherein each of the plurality of fluid outlets in fluid communication with at least one of the plurality of conduits; and
wherein at least some of the plurality of conduits includes at least one of the plurality of sensors positioned therein.

5. The fluid collection assembly of claim 3, wherein the at least one conduit includes an inlet and the at least one sensor is positioned within the inlet.

6. The fluid collection assembly of claim 2, wherein the at least one sensor is positioned within the at least one fluid outlet.

7. The fluid collection assembly of claim 2, wherein the at least one sensor is positioned on the fluid impermeable barrier.

8. The fluid collection assembly of claim 2, wherein the at least one sensor is positioned within the at least one porous material.

9. The fluid collection assembly of claim 2, wherein the fluid impermeable barrier and the at least one porous material define at least one fluid reservoir therebetween, the at least one fluid reservoir positioned adjacent to the at least one fluid outlet, the at least one sensor positioned within the at least one fluid reservoir.

10. The fluid collection assembly of claim 2, wherein the at least one sensor includes at least one of an electrochemical gas sensor, a humistor, a hygrometer, a flow meter, a pH sensor, or two or more electrodes.

11. The fluid collection assembly of claim 2, wherein the at least one opening includes a waist opening and two leg openings.

12. The fluid collection assembly of claim 2, wherein the at least one fluid outlet is spaced from exterior edges of the fluid impermeable barrier and edges of the fluid impermeable barrier that at least partially define the at least one opening.

13. The fluid collection assembly of claim 2, wherein the at least one porous material extending from or near a portion of the waist opening to or near an opposing portion of the waist opening.

14. The fluid collection assembly of claim 13, wherein the at least one porous material includes:
a fluid permeable membrane positioned to contact an individual, the fluid permeable membrane configured to receive and wick bodily fluids away from an individual; and
a fluid permeable support configured to have the bodily fluids flow therein.

15. The fluid collection assembly of claim 14, wherein the fluid permeable support abuts substantially all of an interior surface of the fluid impermeable barrier that is not immediately adjacent to the at least one fluid outlet.

16. A fluid collection system, comprising:
a fluid collection assembly including:
a fluid impermeable barrier at least partially defining:
an interior region;
at least one opening extending through the fluid impermeable barrier to the interior region; and
at least one fluid outlet;
at least one porous material disposed in the interior region generally conforming to at least a portion of the fluid impermeable barrier;
at least one conduit in fluid communication with the interior region via the at least one fluid outlet; and
at least one sensor configured to detect presence of moisture within the fluid collection assembly, the at least one sensor positioned within the at least one conduit, the at least one fluid outlet, or in a portion of the interior region proximate to the at least one fluid outlet;
a vacuum source configured to apply a suction force to the fluid collection assembly to withdraw one or more bodily fluids therefrom; and
a storage container.

17. The fluid collection system of claim 16, wherein the vacuum source is configured to apply the suction force to the fluid collection assembly when the at least one sensor detects the presence of moisture and to cease applying the suction force to the fluid collection assembly when the at least one sensor does not detect the presence of moisture.

18. The fluid collection system of claim 16, further comprising one or more valves between the fluid collection assembly and the vacuum source, the one or more valves configured to open and permit the suction force to be provided to the fluid collection assembly when the at least one sensor detects the presence of moisture and to close and inhibit the suction force from reaching the fluid collection assembly when the at least one sensor does not detect the presence of moisture.

19. The fluid collection system of claim 16, wherein the at least one fluid outlet includes a plurality of fluid outlets, the at least one conduit includes a plurality of conduits, and the at least one sensor includes a plurality of sensors;
wherein each of the plurality of fluid outlets are in fluid communication with at least one of the plurality of conduits; and
wherein at least some of the plurality of conduits includes at least one of the plurality of sensors positioned therein; and
further comprising a plurality of valves, each of the plurality of fluid outlets in fluid communication with at least one of the plurality of valves, the plurality of valves configured to open and permit the suction force to reach one or more of the plurality of fluid outlets when at least one of the plurality of sensors detects the presence of moisture at or near the one or more of the plurality of fluid outlets.

20. The fluid collection system of claim 16, further comprising a controller including at least one processor and memory storage medium, the controller communicably coupled to one or more components of the fluid collection system, the controller configured to only permit the suction force to be provided to the fluid collection assembly when the at least one sensor detects the presence of moisture.

21. The fluid collection system of claim 16, wherein the at least one conduit includes an inlet and the at least one sensor is positioned immediately adjacent to the inlet.
